(19) United States
(12) Patent Application Publication
(10) Pub. No.: US 2015/0290460 A1
(43) Pub. Date: Oct. 15, 2015

(54) Methods for Treating Mild Cognitive Impairment and Alzheimer's Disease

(71) Applicant: Advanced Neuromodulation Systems, Inc. dba St. Jude Neuromodulation Division, Plano, TX (US)

(72) Inventor: Dirk De Ridder, Dunedin (NZ)

(21) Appl. No.: 14/635,961

(22) Filed: Mar. 2, 2015

Related U.S. Application Data
(60) Provisional application No. 61/947,859, filed on Mar. 4, 2014.

(51) Int. Cl. A61N 1/36; A61N 1/05

(52) U.S. Cl. CPC A61N 1/36082; A61N 1/0531; A61N 1/0534; A61N 1/36085; A61N 1/36089; A61N 1/36096; A61N 1/36146; A61N 2001/36039

(57) ABSTRACT

In certain embodiments, a method of treating mild cognitive disorder, early-stage dementia, Alzheimer's disease, or other neurological disorder, comprises: identifying the neurological disorder in a patient; operating an implantable pulse generator to generate electrical pulses; and providing the electrical pulses from the implantable pulse generator to tissue of a target site of the patient's brain, using one or more electrodes of an implantable stimulation lead, to treat the neurological disorder in the patient, wherein the target site is selected from the sites consisting of posterior cingulate cortex (PCC), inferior pariertal area, parahippocampus, precuneus, sub and pregenual anterior cingulate cortex (ACC), and pariertal area sites.

### RELATED APPLICATION

The application claims the benefit of U.S. Provisional Patent Application No. 61/947,859, filed Mar. 4, 2014 which is incorporated herein by reference.

### BACKGROUND

Cognitive disorders are a common type of neurological disorders. For example, dementia is a form of impaired cognition caused by brain dysfunction. The hallmark of most forms of dementia is the disruption of memory performance. Among the several conditions labeled as dementia, the most common are Alzheimer's disease and mild cognitive impairment (MCI), which is a pre-clinical form of Alzheimer's disease. MCI is an intermediate state between normal aging and dementia and is characterized by acquired cognitive deficits, without significant decline in functional activities of daily living. Subjects with MCI and the initial phase of Alzheimer's disease originally present with a predominant deficit in memory function. In more advanced stages of Alzheimer's disease, impairment in additional cognitive domains culminates with a significant decline in quality of life and the inability to perform usual daily activities.

Alzheimer's disease is one of the most common cognitive disorders in humans. Although the defining characteristic of Alzheimer's disease is cognitive impairment, it is often accompanied by mood and behavioral symptoms such as depression, anxiety, irritability, inappropriate behavior, sleep disturbance, psychosis, and agitation. Neuro-imaging and genetic testing have aided in the identification of individuals at increased risk for dementia. However, the measurement of change in cognitive and functional status in, for example, MCI remains challenging because it requires instruments that are more sensitive and specific than those considered adequate for research in dementia. Accordingly, no treatment exists that adequately prevents or cures Alzheimer's disease or MCI.

Similar disease processes are found in other neurological disorders. For example, Parkinson's disease (PD) patients may exhibit dementia during progression of the disorder. Patients suffering from PD-related dementia may experience multiple symptoms including changes in memory, concentration and judgment, difficulty interpreting visual information, visual hallucinations, delusions, depression, irritability and anxiety, and sleep disturbances. Many PD patients with dementia also have plaques and tangles which are hallmark brain changes linked to Alzheimer's disease.

Alzheimer's disease, MCI, and dementia are already a public health problem of enormous proportions. It is estimated that 5 million people currently suffer with Alzheimer's disease in the United States. This figure is likely underestimated due to the high number of unrecognized and undiagnosed patients in the community. By the year 2050, Alzheimer's is projected to affect 14 million people. Moreover, because the prevalence of Alzheimer's disease doubles every 5 years after age 65, the impact of the disease on society tends to increase with the growth of the elderly population. The annual cost in the United States of Alzheimer's Disease alone is approximately $100 billion. Further, there is currently no effective treatment for the memory loss and other cognitive deficits presented by patients with dementia, particularly Alzheimer's disease. Treating Alzheimer's disease tends to be more challenging than other neurological disorders because Alzheimer's largely affects a geriatric population. Oral medications including acetylcholinesterase inhibitors and cholinergic agents are the mainstay treatment for this condition. Nevertheless, the outcome with these agents is modest and tends to decline as the disease progresses.

### BRIEF DESCRIPTION OF THE DRAWINGS

FIGS. 1A-1J illustrate example electrical stimulation leads that may be used to electrically stimulate neuronal tissue.

FIG. 2 depicts an implantable pulse generator that may be programmed to generate stimulation according to one representative embodiment.

FIGS. 3A and 3B illustrate pink noise or 1/f noise. FIG. 3A shows an exemplary pink noise spectrum. FIG. 3B shows an exemplary pink noise generated by a power source, for example an external or implantable generator

FIGS. 4A and 4B illustrate red, brown or Brownian noise or 1/f² noise. FIG. 4A shows an exemplary red or Brown(ian) noise spectrum. FIG. 4B shows an exemplary spectrum generated by a power source, for example an external or implantable generator.

FIGS. 5A-5B illustrate exemplary combinations of 1/f̂β noise. FIG. 5A shows 1/f̂β noise modulated at alpha frequencies and FIG. 5B at imp noise modulated at beta frequencies.

FIG. 6 depicts a stimulation system that can measure or detect given neuronal signals that can be used to modulate the 1/f̂β noise stimulation according to one representative embodiment.

FIG. 7 illustrates a 1/f̂β spectrum at rest for normal and tinnitus patients. β is 2.2 for healthy controls (1.5 for noise-like tinnitus and 1.8 for pure tone tinnitus).

FIG. 8 depicts a stimulation system that can sense and/or monitor sleep stage that can be used to alter therapy.

FIG. 9 shows modules within the memory of FIG. 8.

FIG. 10 depicts respective sets of pulses for desychronization of neuronal activity.

FIG. 11 depicts electrical stimulation leads for cortical stimulation to treat MCI and/or AD in a patient according to representative embodiments of the present invention.

### DETAILED DESCRIPTION

In accordance with different embodiments of the present invention, mild cognitive impairment, early stages of dementia, and/or Alzheimer's disease (AD) may be treated using one or more of methods as discussed herein. In contrast to other known neurostimulation therapies for mild cognitive impairment and/or AD where stimulation of deep brain structures is employed, methods according to embodiments of the present invention stimulate cortical structures. Specifically, in one representative embodiment, the posterior cingulate cortex (PCC) is selected for stimulation to treat patients exhibiting mild cognitive impairment, early-stage dementia, and/or AD. MCI and early-stage dementia treated according to some embodiments may be the result of AD, PD, or any other neurological disorder. In other embodiments, the selected stimulation target may include one or more other areas functionally connected to the PCC, such as the inferior pariertal area, parahippocampus, precuneus, sub and pregenual anterior cingulate cortex (ACC).

Further, stimulation or modulation of the PCC is believed to provide a more effective therapy for mild cognitive impairment, early-stage dementia, and/or AD than stimulation of certain deep brain structures as suggested by others in published literature. In particular, stimulation of deep brain structures (such as hippocampal regions) is not believed to be optimal, because the disease process in AD does not exert its full impact without extension into the neocortex, where correlation is stronger between clinical and pathological observations.

During the spread of neuropathology in AD, neurofibrillary tangles and neurodegeneration first appear in the entorhinal cortex and then in other medial temporal lobe structures. Fibrillary amyloid β deposits and plaques appear in transmodal areas—such as the PCC, the inferior parietal lobule, the lateral temporal lobe, and temporal pole—that maintain reciprocal connections. Spread of neurofibrillary tangles and neurodegeneration is not associated with the spread of fibrillar amyloid β deposition and plaque formation.

Hubs in neuronal structures are areas in the brain that exhibit more connections to other brain areas. Hubs are more active than non-hubs. The high activity of hubs leads to greater neuronal damage and greater deposits of β-amyloid waste products. Thereby, structural connectivity of hubs to other areas decreases in patients. The decrease in structural connectivity may be compensated by greater functional connectivity. However, if functional connectivity compensation cannot follow structural degeneration AD develops.

Hubs are the areas that use most glucose and are those areas with highest centrality. These areas overlap with the default system (i.e., the self-perceptual system). The areas with highest centrality are the PCC and the temporoparietal junction (TPJ). The area with the highest functional connectivity density is the PCC.

AD is characterized by hypersynchronization in parrahippocampal cortex (PHC)/PCC/precuneus, anterior cingulate cortex (ACC) and lateral inferior parietal areas as well as hyposynchronization in other areas (in comparison to healthy control subjects). In rapidly progressing AD, there is an increase in synchronization in time in the PCC and reduction in synchronization in left frontotemporal cortex area.

In representative embodiments, electrical stimulation is provided to patients exhibiting MCI, early-stage dementia, and/or AD. The electrical stimulation increases cell survival, enhances nerve growth, and improves functional connectivity. The electrical stimulation may be provided using one or multiple stimulation methods, including transcranial direct-current stimulation (tDCS), transcranial alternating current stimulation (tACS), transcranial random noise stimulation (tRNS), and implanted electrical stimulation systems.

In representative embodiments, the stimulation applied to the patient is selected to desynchronize the PCC and TPJ. In one embodiment, biparietal tRNS stimulation applies electrical pulses that exhibit Gaussian noise characteristics to desynchronize the PCC and TPJ. In another embodiment, neurofeedback is selected to provide a real-time display of electroencephalography (EEG) signals of Brodmann area 30 to illustrate brain activity for self-regulation of the neuronal activity by the patient.

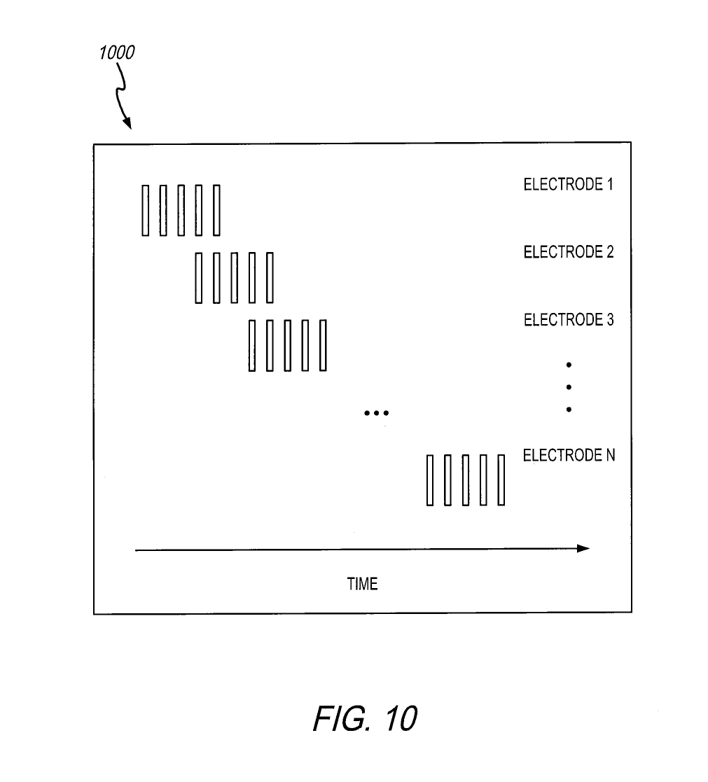

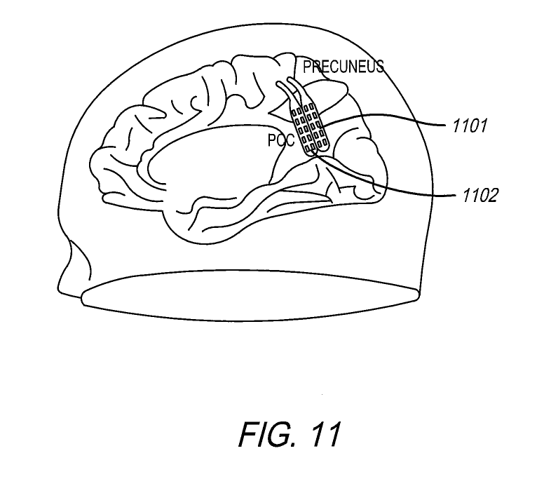

In other representative embodiments, a neurostimulation system is implanted in the patient to desynchronize the PCC and TPJ. The neurostimulation system includes a pulse generator and one or more stimulation leads. The electrodes of the stimulation lead are placed proximate to the dura above the PCC as shown in FIG. 11. Electrical pulses or other electrical signals are provided to the PCC to accomplish the desynchronization. In some embodiments, the electrical signals include pink noise, brown noise, or other noise components as discussed herein. In other embodiments, a “coordinated reset” pulse pattern is applied to cortical areas to desynchronize the PCC and TPJ. Coordinated reset stimulation is shown in stimulation pattern 1000 in FIG. 10 and is described, for example, by Peter A. Tass et al in COORDINATED RESET HAS SUSTAINED AFTEREFFECTS IN PARKINSONIAN MONKEYS, Annals of Neurology, Volume 72, Issue 5, pages 816-820, November 2012, which is incorporated herein by reference. The electrical pulses in coordinated reset pattern 1000 are generated in bursts of pulses with respective bursts being applied to tissue of the patient using different electrodes in a time-offset manner. Coordinated reset stimulation may be applied using, for example, the pulse generator systems described in U.S. Pat. No. 8,433,416 which is incorporated herein by reference.

The following section more generally describes an example of a procedure for treatment using a 1/f̂β noise such as pink noise, red or brown noise or black noise to optimize the following parameters; a set and/or range of stimulation protocols that can most completely eliminate neurological disease/disorder, a set and/or range of stimulation protocols that requires the lowest voltage, and a protocol that maintains treatment efficacy over long periods of time, for example, the protocol can prevent habituation or adaptation and a protocol that is anti-epileptic. Still further, the generated 1/f̂β noise signal can be filtered, combined, or otherwise processed, for example, whereby the generated 1/f̂β noise is utilized as a background signal noise over another signal with a spectral peak at a selected frequency. For example, an alpha peak, beta peak, delta peak and/or theta peak can be added to the 1/f̂β noise.

The peaks can be generated using typical known frequencies or the peaks can be individualized for each patient. Yet further, the 1/f̂β noise can be combined with standard tonic and/or burst stimulation to further enhance the optimization or prevent habituation. Combinations of tonic and/or burst stimulation are known in the art, for example, U.S. Pat. No. 7,734,340, issued Jun. 8, 2010 and U.S. application Ser. No. 12/109,098, filed Apr. 24, 2008, which are incorporated by reference in their entirety.

A noise signal can be described as a signal that is generated according to a random process. In practice, various algorithms (e.g., in software executed on a processor) are employed to simulate a given random process to generate a “pseudo-random” signal where the generated pseudo-random signal possesses similar characteristics with signals corresponding to a corresponding random process. The characteristics of a particular noise signal depend upon the underlying process generating the noise signal. For example, the power spectral density or power distribution in the frequency domain may be employed to characterize the random process and, hence, also characterize a corresponding time-domain noise signal. The classification of the power spectral density of a noise signal may be described in reference to color or color terminology with different types of power spectral densities named after different colors.

According to these conventions, the power spectral density is defined as being inversely proportional to f̂β, where f represents frequency and β is a value selected to characterize the noise signal. The value β can be for example, any real, natural, integer, rational, irrational or complex number. For example, the spectral density for white noise is flat (β=0), for pink noise or flicker noise β=1 and for Brownian or red noise β=2 and black noise is β>2. Suitable non-integer β values about 1 include 0.5, 0.6, 0.7, 0.8, 0.9, 1.0, 1.1, 1.2, 1.3, 1.5, or any values there between for some embodiments. Likewise, suitable non-integer β values about 2 can include 1.6, 1.7, 1.8, 1.9, 2.0, 2.1, 2.2, 2.3, 2.4, 2.5 or any value there between for some embodiments.

Abnormal electrical and/or neural activity is associated with different diseases and disorders in the central and peripheral nervous systems. In addition to a drug regimen or surgical intervention, potential treatments for such diseases and disorders include the implantation of a medical device (for example, an implantable pulse generator (IPG)) in a patient for electrical stimulation of the patient's body tissue. In particular, an implantable medical device may electrically stimulate a target neuronal tissue location by the selective application of controlled electrical input signals to one or more electrodes coupled to or placed in proximity to the patient's neuronal tissue. Such electrical input signals may be applied to the patient's neuronal tissue in order to treat a neurological disease, condition, or disorder.

The response of nonlinear systems to a weak input signal may be optimized by combining the input signal with a non-negligible level of noise or as known in the art as stochastic resonance. For a system to exhibit stochastic resonance there needs to be a threshold that must be exceeded in order to activate the system. When the input signal is not strong enough to exceed the threshold, small amounts of noise added either to the system or the signal may occasionally suffice to trigger activation. Typically this type of phenomenon is associated with white noise.

Over time, a repetitive electrical stimulation signal, such as typical electrical stimulation performed today, that is dissimilar to the brain's own naturally-occurring signals may become less effective as the brain “filters out” or “ignores” the signal. Hence, a problem with standard electrical stimulation parameters used today is habituation because the electrical stimulation parameters result in a repetitive electrical signal and thus, the brain habituates to the signal or adapts. It is believed that naturally-occurring signals within the human brain closely resemble 1/f̂β noise. Because of this, the efficacy of electrical stimulation signals applied to neuronal tissue is improved by making those signals comport as closely as possible to the brain's own signals. Such a signal may be less likely to lose effectiveness over time. One way to comport an electrical stimulation signal to resemble the brain's own signals is to utilize a stimulation paradigm that resembles that of the brain's normal signals, for example convert the pink noise spectrum into electrical stimulation signals that can be applied to the desired neuronal tissue at a desired pattern, frequency, amplitude such that it maintains parameters associated with 1/f̂β noise spectrum. To further modulate the 1/f̂β noise stimulation paradigm, add specific peak frequencies to the 1/f̂β noise stimulation paradigm that are known or associated with given brain areas, for example, add an alpha frequency peak to stimulate primary and secondary cortical areas; add a beta frequency peak to stimulate association cortical areas, such as frontal cortex; add a theta frequency peak to stimulate the cingulate, hippocampus, amygdala. Suitable peak frequencies that may be added to the 1/f̂β noise stimulation paradigm can be obtained from the individual by EEG or MEG measurements or any other measurement to obtain the individual peak frequency or the frequencies can be obtained from a database, for example a database containing a list of given frequencies and spectral structures for a brain structure or brain area. The frequency for each brain area, for example, each Brodmann area can be easily calculated by defining a Brodmann area in source space and performing a spectral analysis for that area using any software (i.e., sLORETA) to perform source analysis.

Still further, the 1/f̂β noise stimulation paradigm can be modified by using multiple electrodes, for example, the stimulation paradigm is either sequentially cycled or randomly cycled through the electrodes upon the stimulation lead.

The 1/f̂β noise can also be selected to specifically activate or inactivate a brain area or brain network, i.e., it can be chosen so as to not be normalizing, but to be non-physiological as to compensate for overactivity or hypoactivity, followed at a later stage with normal physiological 1/f̂β noise stimulation parameters. For example during sleep, changing the spectral characteristics of the 1/f̂β noise may be advantageous physiologically.

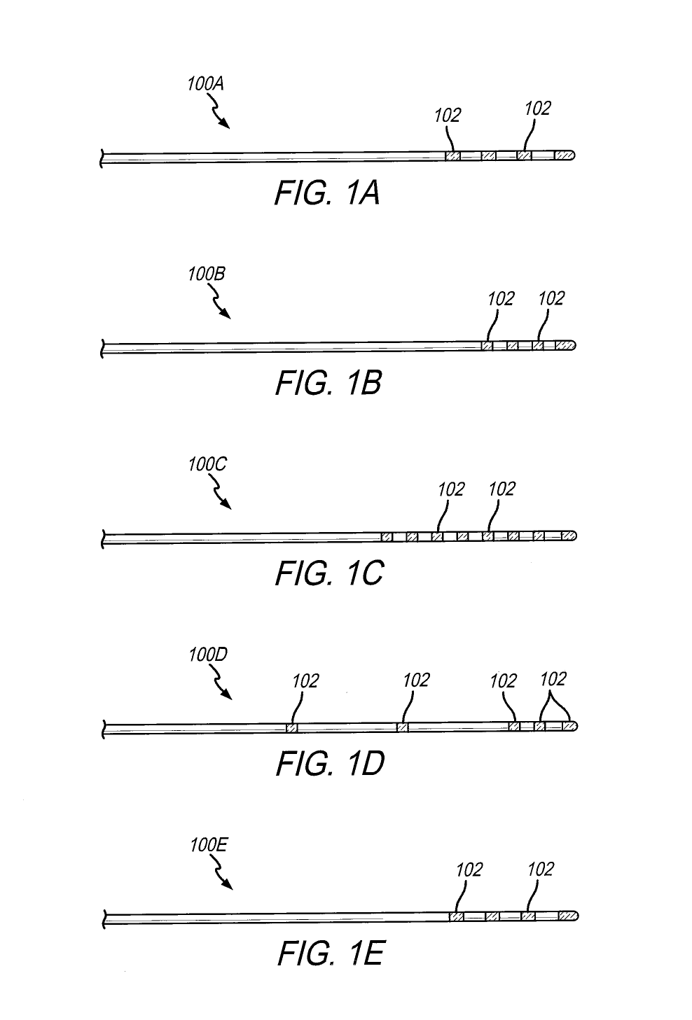

One or more stimulation leads 100, as shown in FIGS. 1A-1J are implanted such that one or more stimulation electrodes 102 of each stimulation lead 200 are positioned or disposed near, adjacent to, directly on or onto, proximate to, directly in or into or within the target tissue or predetermined site. The leads shown in FIG. 1 are exemplary of many commercially available leads, such as deep brain leads, percutaneous leads, paddle leads, etc. Examples of commercially available stimulation leads includes a percutaneous OCTRODE™ lead or laminotomy or paddle leads or paddle structures such as PENTA™ lead or LAMITRODE 44 ™ lead all manufactured by St. Jude Medical. For the purposes described herein and as those skilled in the art will recognize, when an embedded stimulation system, such as the Bion™, is used, it is positioned similar to positioning the lead 100.

According to stimulation of the PCC or other sites as described herein, any of the stimulation leads illustrated in FIGS. 1A-1J can be implanted for cortical stimulation, as well as any other cortical electrode or electrode array. Techniques for implanting stimulation electrodes are well known by those of skill in the art. For implanting conventional cortical electrodes, it typically requires a craniotomy under general anesthesia to remove a suitably sized window in the skull. A pilot hole can be formed through at least part of the thickness of the patient's skull adjacent a selected or predetermined site. In certain embodiments, the pilot hole can be used as a monitoring site.

The location of the pilot hole (and, ultimately the electrode received therein) can be selected in a variety of fashions, for example, the physician may use anatomical landmarks, e.g., cranial landmarks such as the bregma or the sagittal suture, to guide placement and orientation of the pilot hole or the physician may use a surgical navigation system. Navigation systems may employ real-time imaging and/or proximity detection to guide a physician in placing the pilot hole and in placing the electrode in the pilot hole. In some systems, fiducials are positioned on the patient's scalp or skull prior to imaging and those fiducials are used as reference points in subsequent implantation. In other systems, real-time MRI or the like may be employed instead of or in conjunction with such fiducials. A number of suitable navigation systems are commercially available, such as the STEALTHSTATION TREON TGS sold by Medtronic Surgical Navigation Technologies of Louisville, Colo., U.S.

Once the pilot hole is formed, the threaded stimulation lead may be advanced along the pilot hole until the contact surface electrically contacts a desired portion of the patient's brain. If the stimulation lead is intended to be positioned epidurally, this may comprise relatively atraumatically contacting the dura mater; if the electrode is to contact a site on the cerebral cortex, the electrode will be advanced to extend through the dura mater. Thus, the lead may be placed epidurally or subdurally for cortical stimulation.

Conventional neuromodulation devices can be modified to apply a 1/f̂β noise stimulation, or 1/f̂β noise stimulation in combination with individual peak frequencies (e.g., alpha, beta, theta and delta) or combination of 1/f̂β noise stimulation combined with burst or tonic stimulation to nerve tissue of a patient by modifying the software instructions and/or stimulation parameters stored in the devices. Specifically, conventional neuromodulation devices typically include a microprocessor and a pulse generation module. The pulse generation module generates the electrical pulses according to a defined pulse width and pulse amplitude and applies the electrical pulses to defined electrodes through switching circuitry and the wires of a stimulation lead. The microprocessor controls the operations of the pulse generation module according to software instructions stored in the device and accompanying stimulation parameters. Examples of commercially available neuromodulation devices that can be modified according to some embodiments include the EON™ or EON mini™, manufactured by St. Jude Medical. Other neuromodulation devices that may be modified can include, LIBRA™ or BRIO™ manufactured by St. Jude Medical.

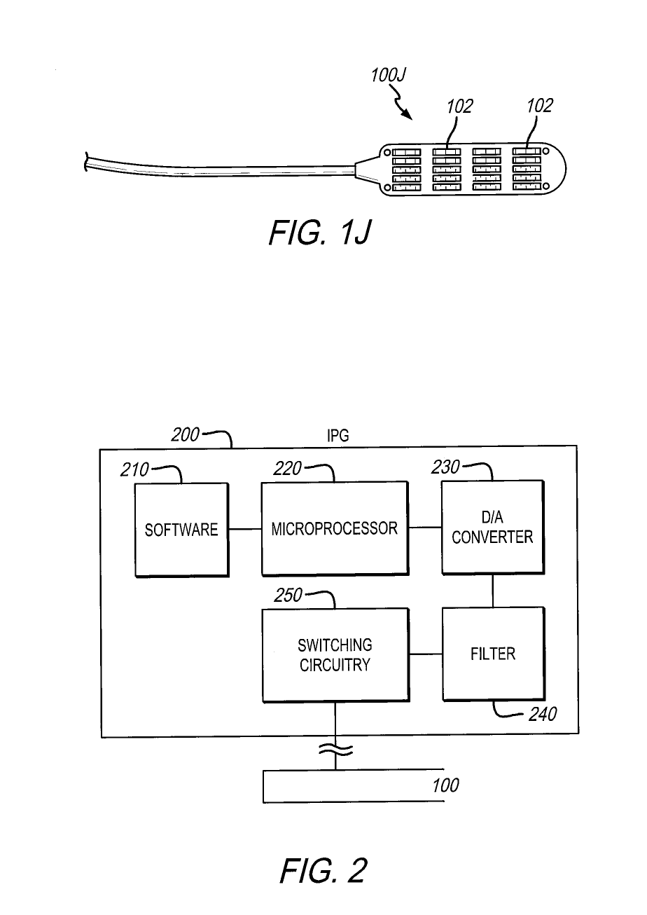

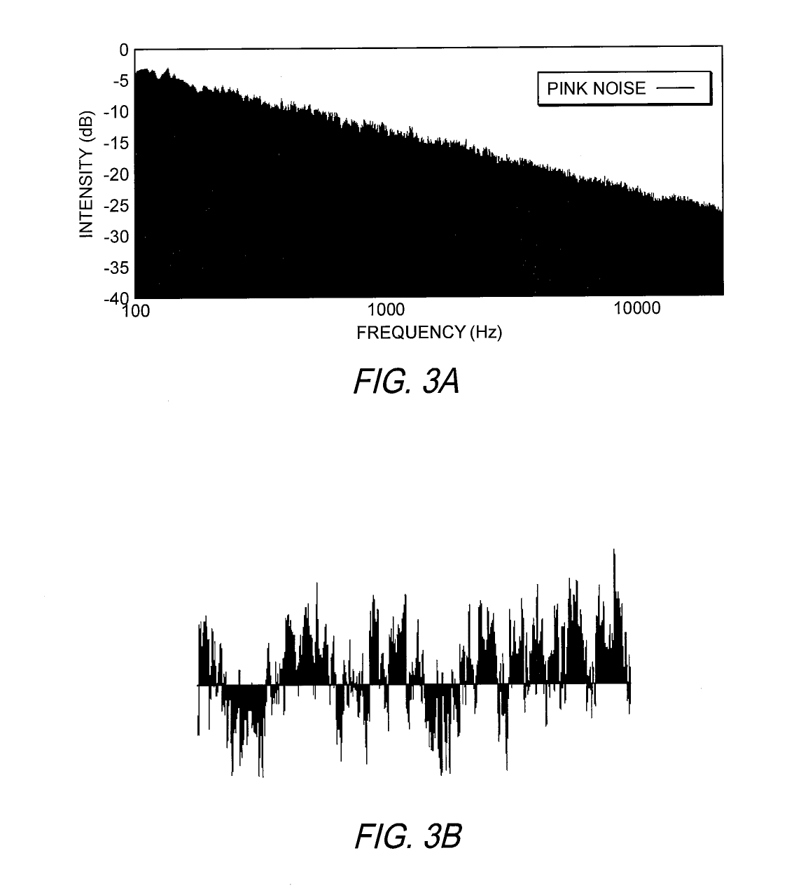

These neuromodulation devices can be adapted by modifying the software instructions provided within the neuromodulation devices used to control the operations of the devices. In some embodiments, software is provided within the neuromodulation device to retrieve or generate a stream of digital values that define a waveform according to the desired power spectral density. This stream of values is then employed to control the amplitude of successive stimulation pulses generated by the neurostimulation device. The software may include a pseudo-random number generator according to known algorithms to generate the stream of digital values. Alternatively, one or more streams of digital values having the desired power spectral density may be generated offline and stored in memory of the neuromodulation device (in a compressed or other suitable format). The software of the neuromodulation device may retrieve the values from memory for control of the amplitude of the output pulses of the neuromodulation device. Alternatively, an external conventional neuromodulation devices can be used (for example, the DS8000™ digital stimulator available from World Precision Instruments) to generate the desired electrical stimulation. For example, a custom waveform may be generated offline on a personal computer and imported into the digital stimulator for pulse generation. Signal parameters may be inputted, such as 1/f̂β noise spectrum, for example FIG. 3A or FIG. 4A, into suitable waveform generating software to generate the stream of digital values. Alternatively, depending upon the capabilities of the external digital stimulator, the stream of digital values may be calculated on board the processor of the external digital stimulator.

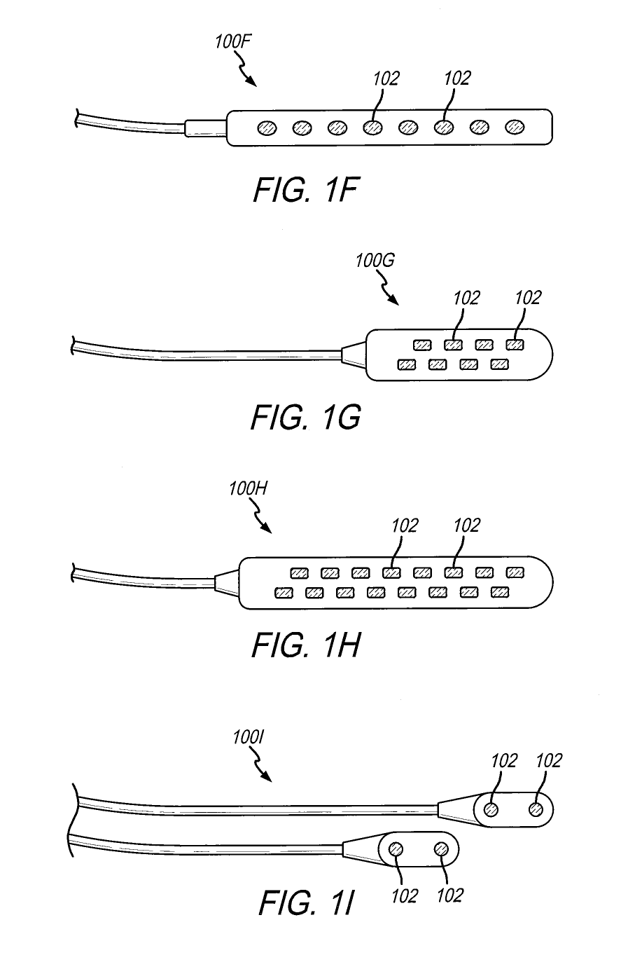

FIG. 2 depicts an exemplary neuromodulation device that can be used to provide the desired stimulation. Signal parameters are inputted, such as 1/f̂β noise spectrum, for example FIG. 3A or FIG. 4A, into the software or memory 210 and the desired wave pattern or signals are generated using microprocessor 220. A standard digital-to-analog converter 230 receives the calculated digital signals and generates analog output pulses corresponding to the values of the digital signals. The generated output pulses may be outputted from the neuromodulation device through an output capacitor. Optionally, any suitable filter 240 can be used to smooth or shape the signals; however, unsmoothed or unfiltered signals can be transmitted to the switching circuitry 250 which provides the signals to the electrodes 100 thereby stimulating the neuronal tissue using the desired 1/f̂β noise stimulation pattern. As an example, the stimulator design disclosed in U.S. Pat. No. 7,715,912 may be employed to generate stimulation pulses according to the desired stimulation pattern. FIGS. 3B and 4B illustrate exemplary waveforms generated by external generators and provided to the electrodes to stimulate neuronal tissue with the 1/f̂β noise stimulation pattern.

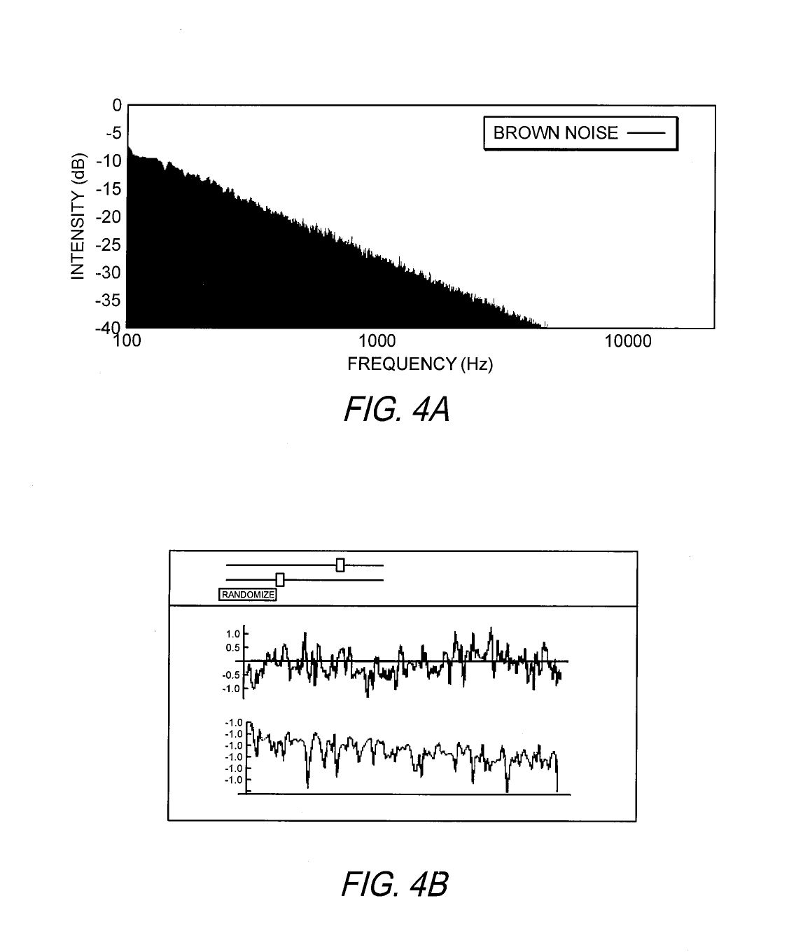

In addition to providing a stimulation waveform similar to that of 1/f̂β noise spectrum; it may be desirable to modify the 1/f̂β noise waveform stimulation pattern. Such modifications can utilize the addition of peak frequencies, such as the addition of an alpha, beta, theta, and/or delta peaks to the 1/f̂β noise spectrum waveform, see for example, FIGS. 5A and 5B. Such frequency peaks can be obtained by using standard peaks or individualizing the frequency peaks. Such information can be communicated to the microprocessor 220 via the software component 210. Thus, the data communicated can comprise standard frequency peaks or comprise individualized frequency peaks or patient specific. The patient specific frequency peaks can be obtained off-line or in real time or on-line, for example prior to implantation or at any time point after implantation, for example, during the initial programming of the IPG. Any suitable signal processing technique may be employed to add the appropriate spectral peaks. For example, a suitable filter may be applied to the noise signal. Alternatively, a separate signal may be generated with a spectral peak about the desired frequency and the separate signal may be added to or superimposed on the noise signal.

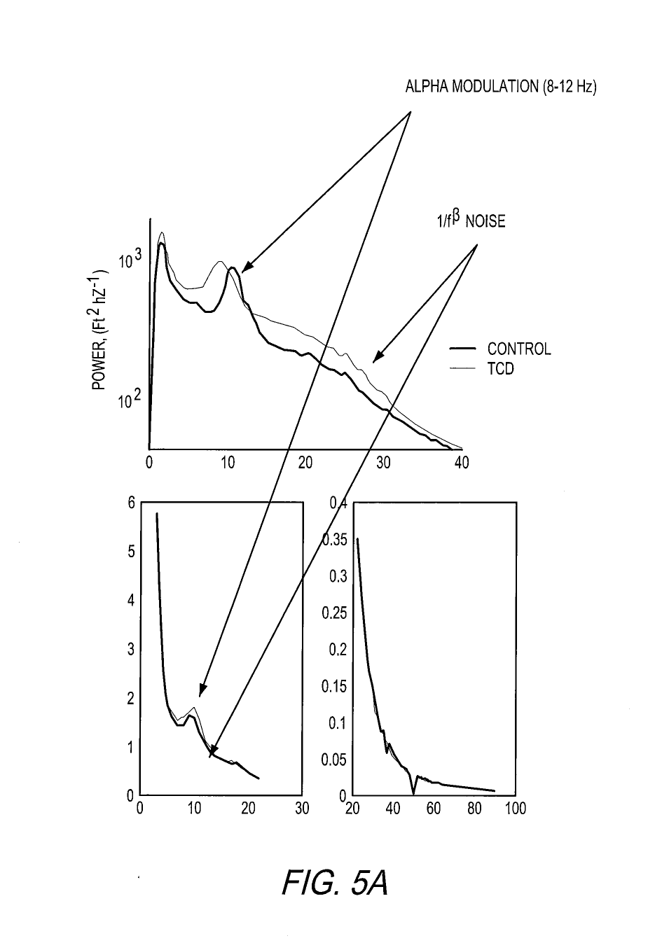

With reference to FIG. 6, with electrodes disposed near, adjacent to, directly next to or within the target neuronal tissue, for example, brain tissue, some representative embodiments utilize the detection and analysis of neuronal activity, such as EEG measurements. Specifically, terminals of the lead, such as an EEG lead, may be coupled using respective conductors 601 to external controller that contains suitable circuitry to analyze neuronal activity, for example, an EEG analyzer can be included in the external controller in which the analyzer functions are adapted to receive EEG signals from the electrodes and process the EEG signals to identify frequency peaks, such as LORETA software can be used. Further signal processing may occur on a suitable computer platform within the external controller using available signal processing. The computer platform may include suitable signal processing algorithms (e.g., time domain segmentation, FFT processing, windowing, logarithmic transforms, etc.). Further platforms or algorithms to modify the signals are included in the modification algorithms (e.g., envelope modification, etc). User interface software may be used to present the processed neuronal activity (i.e., specific peak frequency) and combine a specific peak frequency with the 1/f̂β noise stimulation waveform patterns to the transmitter 603 which then transmits, for example, via radio frequency to the IPG 604 which is adapted to provide the 1/f̂β noise stimulation waveform patterns with the peak frequency to achieve stimulation of the target neuronal tissue via electrode 100. This procedure can be performed on-line or off-line. Additionally, IPG 604 preferably comprises circuitry such as an analog-to-digital (AD) converter, switching circuitry, amplification circuitry, transmitters, and/or filtering circuitry.

Still further, it may be desirable to utilize another implantable device that is capable of performing the functions of the external controller. Thus, those of skill in the art can modify an implantable device such that it is capable of detecting/sampling and processing of the signals representative of the neuronal activity/EEG activity. Such a device may include a microprocessor that is capable of performing these activities as well as a transmitter such that the signals can be transmitted via radiofrequency to another implantable device, such as described above in FIG. 6 that is capable of generating the desired signal to the target tissue. Thus, an EEG lead is placed or positioned near the target brain tissue via methods known to those of skill in the art. The EEG lead detects neuronal activity which is relayed to the processor that possesses sufficient computational capacity to collect the information obtained from the EEG electrode, process it to obtain the respective frequency peak desired and/or modulate the frequency peaks and transmit the frequency to an RF transmitter that transmits the respective information to microprocessor located in the stimulation IPG.

Another means to modify the 1/f̂β waveform stimulation pattern is to combine it with tonic stimulation or burst stimulation as described in U.S. Pat. No. 7,734,340, issued Jun. 8, 2010 and U.S. patent application Ser. No. 12/109,098, filed Apr. 24, 2008, both of which are incorporated by reference in their entirety. Thus, a neuromodulation device can be implemented to apply either burst or tonic stimulation using a digital signal processor and one or several digital-to-analog converters. The burst stimulus and/or tonic stimulus waveform could be defined in memory and applied to the digital-to-analog converter(s) for application through electrodes of the medical lead. The digital signal processor could scale the various portions of the waveform in amplitude and within the time domain (e.g., for the various intervals) according to the various burst and/or tonic parameters. A doctor, the patient, or another user of stimulation source may directly or indirectly input stimulation parameters to specify or modify the nature of the stimulation provided.

Thus, a microprocessor and suitable software instructions to implement the appropriate system control can be used to control the burst and/or tonic stimulation in combination with the 1/f̂β stimulation. The processor can be programmed to use “multi-stim set programs” which are known in the art. A “stim set” refers to a set of parameters which define a pulse to be generated. For example, a stim set defines pulse amplitude, a pulse width, a pulse delay, and an electrode combination. The pulse amplitude refers to the amplitude for a given pulse and the pulse width refers to the duration of the pulse. The pulse delay represents an amount of delay to occur after the generation of the pulse (equivalently, an amount of delay could be defined to occur before the generation of a pulse). The amount of delay represents an amount of time when no pulse generation occurs. The electrode combination defines the polarities for each output which, thereby, controls how a pulse is applied via electrodes of a stimulation lead. Other pulse parameters could be defined for each stim set such as pulse type, repetition parameters, etc. Still further, the 1/f̂β waveform stimulation pattern alone or in combination with either burst and/or tonic may be implemented such that the stimulation occurs either sequentially, randomly or pseudo-sequentially over multiple poles or electrodes on the stimulation lead.

In certain embodiments, the stimulation parameters may comprise a burst stimulation having a frequency in the range of about 1 Hz to about 300 Hz in combination with a tonic stimulation having a frequency in the range of about 1 Hz to about 300 Hz. Those of skill in the art realize that the frequencies can be altered depending upon the capabilities of the IPGs that are utilized. More particularly, the burst stimulation may be at about 6, 18, 40, 60, 80, 100, 150, 200, 250 or 300 Hz consisting of 5 spikes with 1ms pulse width, 1 ms interspike interval in combination with 1/f̂β signals interspersed between or around the burst or prior to or after the burst or in any variation thereof depending upon the efficacy of treatment. Still further, 1/f̂β signals or stimulation paradigm as described herein may be used in combination with about 6, 18, 40, 60, 80, 100, 150, 200, 250, 300 Hz tonic stimulation interspersed between or around the 1/f̂β signals or stimulation paradigm, or any variation thereof depending upon the efficacy of treatment and the capabilities of the IPG.

Still further, those of skill in the art recognize that burst firing refers to an action potential that is a burst of high frequency spikes (300-1000 Hz) (Beurrier et al., 1999). Burst firing acts in a non-linear fashion with a summation effect of each spike and tonic firing refers to an action potential that occurs in a linear fashion.

Yet further, burst can refer to a period in a spike train that has a much higher discharge rate than surrounding periods in the spike train (N. Urbain et al., 2002). Thus, burst can refer to a plurality of groups of spike pulses. A burst is a train of action potentials that, possibly, occurs during a ‘plateau’ or ‘active phase’, followed by a period of relative quiescence called the ‘silent phase’ (Nunemaker, Cellscience Reviews Vol 2 No. 1, 2005.) Thus, a burst comprises spikes having an inter-spike interval in which the spikes are separated by 0.5 milliseconds to about 100 milliseconds. Those of skill in the art realize that the inter-spike interval can be longer or shorter. Yet further, those of skill in the art also realize that the spike rate within the burst does not necessarily occur at a fixed rate; this rate can be variable. A spike refers to an action potential. Yet further, a “burst spike” refers to a spike that is preceded or followed by another spike within a short time interval (Matveev, 2000), in other words, there is an inter-spike interval, in which this interval is generally about 100 ms but can be shorter or longer, for example 0.5 milliseconds.

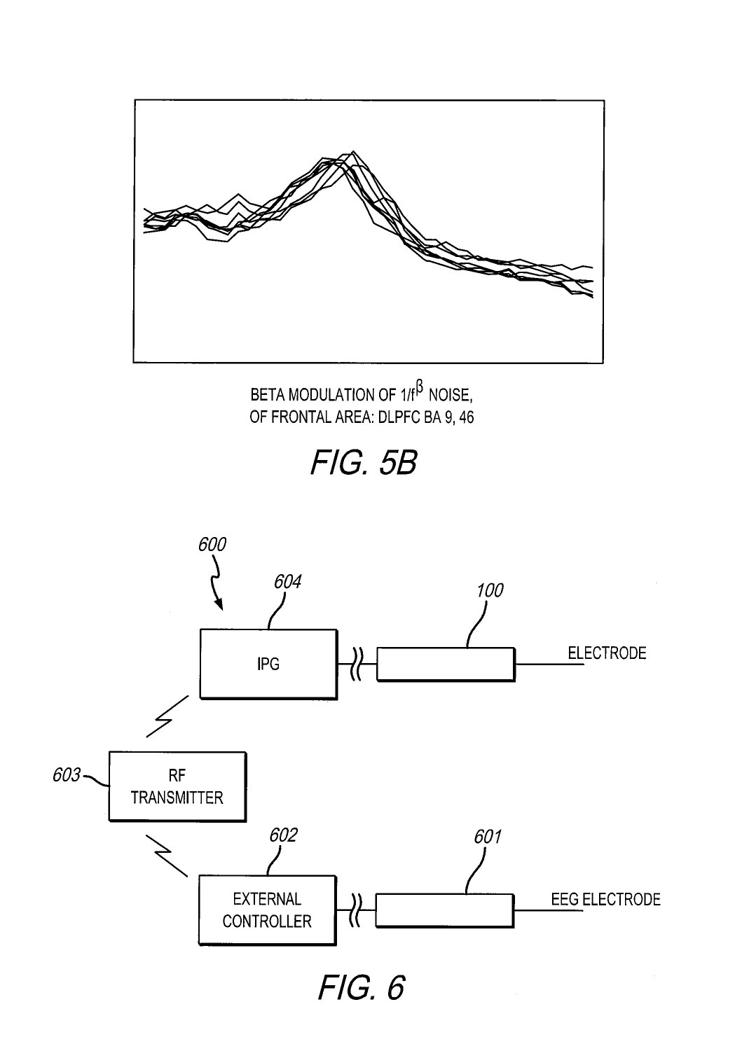

Still further, it may be of interest to use a system that includes a processor that determines whether the patient is in a sleep state, and controls therapy based upon the sleep state. The sleep state may be relevant for 1/f̂β noise stimulation therapy if during a given sleep stage the patient's frequency spectrum changes, for example, the high frequency is adjusted such that the spectrum moves from pink or brown noise to black noise. For example, FIG. 7 shows 1/f² (brown noise) activity at rest in a human tinnitus patient and in normal patients. At rest, the brain has an activity at 1/f² (brown noise). Stimulation applied to the PCC or other suitable stimulationsite may vary the noise characteristics in this manner depending upon the active state or sleep/rest state of the patient. The noise parameter β may be increased from a first value at an active state to a different value for a sleep state.

As referred to herein, the sleep state may refer to a state in which patient is intending on sleeping (e.g., initiating thoughts of sleep), is at rest, is attempting to sleep or has initiated sleep and is currently sleeping. In addition, the processor may determine a sleep stage of the sleep state based on a biosignal detected within brain the patient and control therapy delivery to patient based on a determined sleep stage. Examples of biosignals include, but are not limited to, electrical signals generated from local field potentials within one or more regions of brain, such as, but not limited to, an electroencephalogram (EEG) signal or an electrocorticogram (ECOG) signal. The biosignals that are detected may be detected within the same tissue site of brain as the target tissue site for delivery of electrical stimulation. In other examples, the biosignals may be detected within another tissue site.

Within a sleep state, the patient may be within one of a plurality of sleep stages. Example sleep stages include, for example, Stage 1 (also referred to as Stage N1 or S1), Stage 2 (also referred to as Stage N2 or S2), Deep Sleep (also referred to as slow wave sleep), and rapid eye movement (REM). The Deep Sleep stage may include multiple sleep stages, such as Stage N3 (also referred to as Stage S3) and Stage N4 (also referred to as Stage S4). In some cases, the patient may cycle through the Stage 1, Stage 2, Deep Sleep, REM sleep stages more than once during a sleep state. The Stage 1, Stage 2, and Deep Sleep stages may be considered non-REM (NREM) sleep stages.

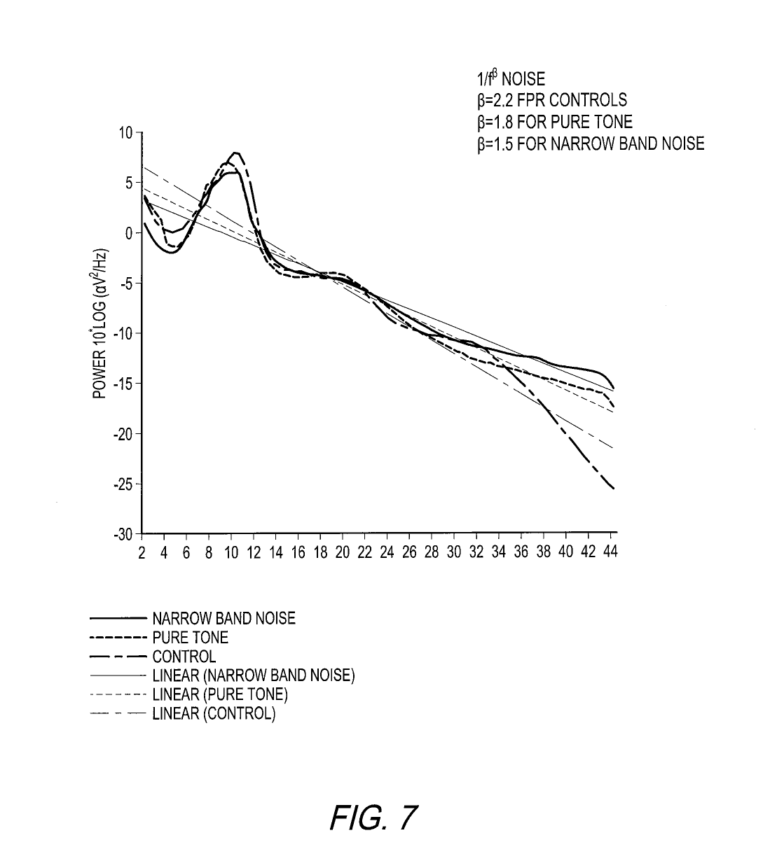

FIG. 8 shows an exemplary implantable neuromodulation device 800 that can be used to determine a stage of sleep and adjust therapy. For example, the device may include, processor 802, memory 801, stimulation generator 804, sensing module 805, telemetry module 806, and sleep stage detection module 803. Although sleep stage detection module 803 is shown to be a part of processor 802 in FIG. 7, in other examples, sleep stage detection module 803 and processor 802 may be separate components and may be electrically coupled, e.g., via a wired or wireless connection.

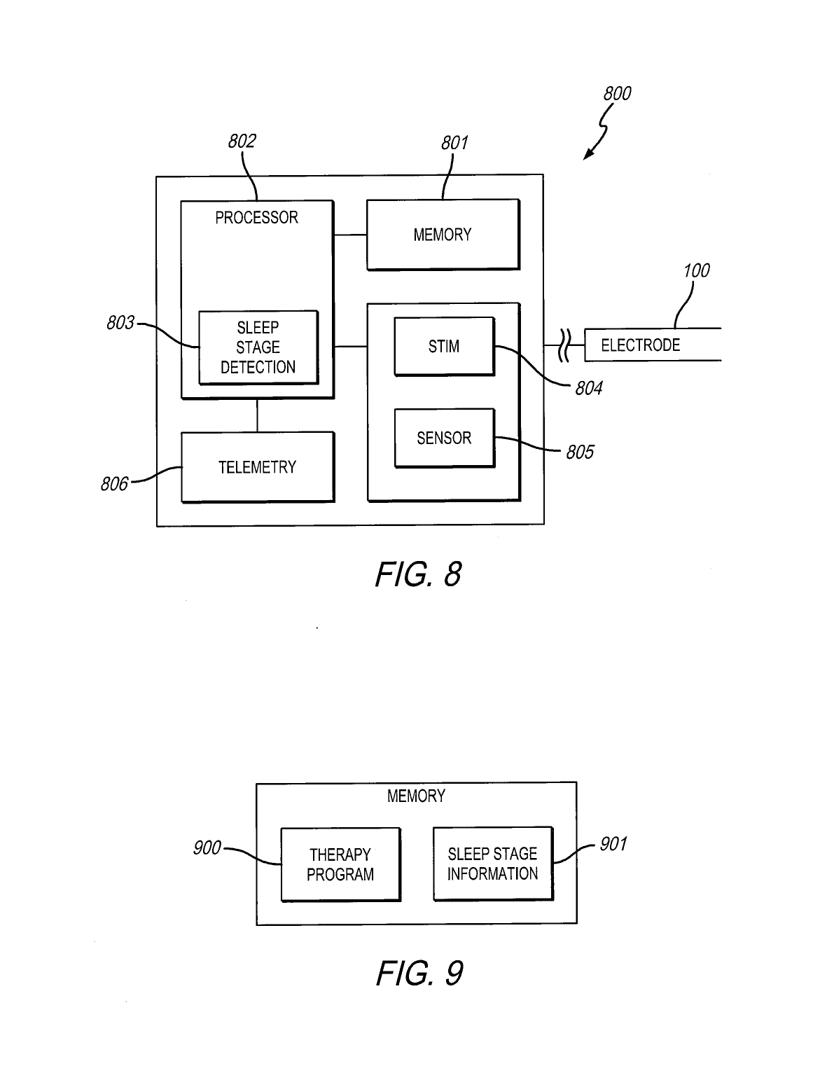

Memory 801, as shown in FIG. 9, may include any volatile or non-volatile media, such as a random access memory (RAM), read only memory (ROM), non-volatile RAM (NVRAM), electrically erasable programmable ROM (EEPROM), flash memory, and the like. Memory 801 may store instructions for execution by processor 802 and information defining therapy delivery for the patient, such as, but not limited to, therapy programs or therapy program groups, information associating therapy programs with one or more sleep stages, thresholds or other information used to detect sleep stages based on biosignals, and any other information regarding therapy of the patient. Therapy information may be recorded in memory 801 for long-term storage and retrieval by a user. As described in further detail with reference to FIG. 9, memory 801 may include separate memories for storing information, such as separate memories for therapy programs 900, and sleep stage information 901. Yet further, other memories that may be stored may include patient information, such as information relating to specific peak frequencies, or information relating to 1/f̂β stimulation.

It is also envisaged that the recording electrode can be used to record or detect sleep stage or when a subject is not in a sleep stage, the recording electrode can be used to detect a change in the normal spectral composition of the noise and adjust the parameters of the stimulation therapy, for example, adjust the stimulation factors such as drowsiness, stress, depression, excitement, arousal, alcohol or other drug intake etc. In other embodiments, other physiological signals (individually or in combination) may be monitored to determine whether a patient is in an active state or a rest/sleep state including, but not limited to, heart rate, respiration rate, EEG signals, EMG signals, posture-related signals (e.g., as determined by sensors in the implanted device), etc. In other embodiments, a timer mechanism may be employed to control application of different stimulation programs according to pre-defined time intervals for the patient to correspond to the active states and sleep/rest states.

Electrical stimulation and/or desynchronization of neuronal activity between the PCC and the TPJ as described herein is believed to improve patient functioning in patients exhibiting MCI and possibly slow or halt progression of AD. Electrical stimulation of the PCC or other suitable stimulation site is believed to increase cell survival, enhances nerve growth, and improves functional connectivity. Improvements in patient functioning may involve improved cognitive functions, improved memory performance, and/or improved psychological well-being of patients. A reduction in symptoms from mild cognitive impairment and/or early-stage dementia is likely to result from stimulation of the PCC and other suitable stimulation sites.

For purposes of this invention, beneficial or desired clinical results include, but are not limited to, alleviation of symptoms, improvement of symptoms, diminishment of extent of disease, stabilized (i.e., not worsening) state of disease, delay or slowing of disease progression, amelioration or palliation of the disease state, and remission (whether partial or total), whether objective or subjective. The improvement is any observable or measurable improvement. Thus, one of skill in the art realizes that a treatment may improve the patient condition, but may not be a complete cure of the disease.

If the subject's neurological disorder/disease has not sufficiently improved, or if the reduction of the neurological disorder/disease is determined to be incomplete or inadequate during an intra-implantation trial stimulation procedure, stimulation lead may be moved incrementally or even re-implanted, one or more stimulation parameters may be adjusted, or both of these modifications may be made and repeated until at least one symptom associated with the neurological disorder/disease has improved.

In certain embodiments, it may be desirable for the patient to control the therapy to optimize the operating parameters to achieve increased or optimized the treatment. For example, the clinician can alter the pulse frequency, pulse amplitude and pulse width using a hand held device that communicates with the IPG using wireless communication protocols. Once the operating parameters have been altered, the parameters can be stored in a memory device to be retrieved by either the patient or the clinician. Yet further, particular parameter settings and changes therein may be correlated with particular times and days to form a patient therapy profile that can be stored in a memory device.

Although some embodiments are directed to treating AD and/or cognitive impairment, alternative embodiments treat other neurological disorders. Other embodiments of neurostimulation for treating neurological disorders may stimulate neuronal “hubs” including posterior cingulate cortex (PCC), inferior pariertal area, parahippocampus, precuneus, sub and pregenual anterior cingulate cortex (ACC), and pariertal area sites. Stimulation of one or more of these sites may include any of the stimulation patterns described herein in regard to AD and/or cognitive impairment. Suitable neurological disorders for treatment by neurostimulation include addiction, anxiety, distress, major depression, attention deficit hyperactivity disorder (ADHD), schizophrenia, traumatic brain injury, eating disorders including obesity and anorexia nervosa, disorders of consciousness, autistic spectrum disorder as examples. In some specific embodiments, a single site (a “hub”) is stimulated using a single set of electrodes with burst stimulation; such burst stimulation of a hub neuronal site is believed to increase functional connectivity to treat a neurological disorder in the patient. In another embodiment, two separate non-hub sites are simultaneously stimulated using burst stimulation; the two separate non-hub sites “wire” together in response to the coordinated stimulation; and this process increases functionality connectivity to treat a neurological disorder in the patient.

In another embodiment, when there is excessive connectivity to the posterior cingulate, such as in distress, anxiety or addiction, noise stimulation might be the preferred stimulation pattern in order to decrease the connectivity.

In some embodiments, respective bursts being applied to tissue of the patient using different electrodes in a time-offset manner to synchronize or desynchronize neuronal activity in the patient. In some embodiments, the electrical pulses are generated in a noisy pattern being applied to tissue of the patient using different electrodes in a time-offset manner to synchronize or desynchronize neuronal activity in the patient.

As will be recognized by those skilled in the art, the innovative concepts described in the present application can be modified and varied over a wide range of applications. Accordingly, the scope of patented subject matter should not be limited to any of the specific exemplary teachings discussed above, but is instead defined by the following claims.

1. A method of treating mild cognitive disorder, early-stage dementia, or Alzheimer's disease, comprising:
identifying early-stage dementia, mild cognitive impairment, or Alzheimer's disease in a patient;
operating an implantable pulse generator to generate electrical pulses; and
providing the electrical pulses from the implantable pulse generator to tissue of a target site of the patient's brain, using one or more electrodes of an implantable stimulation lead, to treat early-stage dementia, mild cognitive impairment, or Alzheimer's disease in the patient, wherein the target site is selected from the sites consisting of posterior cingulate cortex (PCC), inferior pariertal area, parahippocampus, precuneus, sub and pregenual anterior cingulate cortex (ACC), and pariertal area sites.

2. The method of claim 1 wherein the electrodes of the stimulation lead are disposed exterior to the dura above the target site of the patient.

3. The method of claim 1 wherein the electrical pulses are generated to exhibit pink noise, brown noise, or black noise.

4. The method of claim 1 wherein the electrical pulses are generated in bursts of pulses with respective bursts being applied to tissue of the patient using different electrodes in a time-offset manner to synchronize or desynchronize neuronal activity in the patient.

5. The method of claim 1 wherein the electrical pulses are generated in a noisy pattern being applied to tissue of the patient using different electrodes in a time-offset manner to synchronize or desynchronize neuronal activity in the patient.

6. The method of claim 1 further comprising:
programming the implantable pulse generator to include a first stimulation program and a second stimulation program, wherein (i) the implantable pulse generator is operable to generate one or more sequences of pulses according to a variable noise component having a shaped spectral power density controlled by a noise parameter, (ii) the first stimulation program is for an active state of the patient, B1 is a value selected for the noise parameter for the first stimulation program, and a spectral profile defined by the first stimulation program is related to 1/f̂B1 where f represents frequency, (iii) the second stimulation program is for a rest state of the patient, B2 is a value selected for the noise parameter for the second stimulation program, and a spectral profile defined by the second stimulation program is related to 1/f̂B2 where f represents frequency, and (iv) B1 is greater than or equal to 1 and B2 is greater than B1 such that the second stimulation program exhibits lower power density at higher frequencies than the first stimulation program; and
wherein the operating the implantable pulse generator comprises: generating electrical pulses according to the first stimulation program during an active state of the patient and generating electrical pulses according to the second stimulation program during a rest state of the patient.

7. A method of treating a neurological condition, comprising:
identifying the neurological condition in a patient, wherein the neurological condition is selected from the list consisting of addiction, anxiety, distress, major depression, attention deficit hyperactivity disorder (ADHD), schizophrenia, traumatic brain injury, an eating disorder, a disorder of consciousness, and an autistic spectrum disorder;
operating an implantable pulse generator to generate electrical pulses; and
providing the electrical pulses from the implantable pulse generator to tissue of a target site of the patient's brain, using one or more electrodes of an implantable stimulation lead, to treat the neurological condition, wherein the target site is selected from the sites consisting of posterior cingulate cortex (PCC), inferior pariertal area, parahippocampus, precuneus, sub and pregenual anterior cingulate cortex (ACC), and pariertal area sites.

8. The method of claim 7 wherein the electrodes of the stimulation lead are disposed exterior to the dura above the target site of the patient.

9. The method of claim 7 wherein the electrical pulses are generated to exhibit pink noise, brown noise, or black noise.

10. The method of claim 7 wherein the electrical pulses are generated in bursts of pulses with respective bursts being applied to tissue of the patient using different electrodes in a time-offset manner to desychronize neuronal activity in the patient.

11. The method of claim 7 further comprising:
programming the implantable pulse generator to include a first stimulation program and a second stimulation program, wherein (i) the implantable pulse generator is operable to generate one or more sequences of pulses according to a variable noise component having a shaped spectral power density controlled by a noise parameter, (ii) the first stimulation program is for an active state of the patient, B1 is a value selected for the noise parameter for the first stimulation program, and a spectral profile defined by the first stimulation program is related to 1/f̂B1 where f represents frequency, (iii) the second stimulation program is for a rest state of the patient, B2 is a value selected for the noise parameter for the second stimulation program, and a spectral profile defined by the second stimulation program is related to 1/f̂ B2 where f represents frequency, and (iv) B1 is greater than or equal to 1 and B2 is greater than B1 such that the second stimulation program exhibits lower power density at higher frequencies than the first stimulation program; and
wherein the operating the implantable pulse generator comprises: generating electrical pulses according to the first stimulation program during an active state of the patient and generating electrical pulses according to the second stimulation program during a rest state of the patient.

12. A method of treating a neurological disorder, comprising:
identifying the neurological disorder in the patient, wherein the neurological disorder is selected from the list consisting of: addiction, anxiety, distress, major depression, attention deficit hyperactivity disorder (ADHD), schizophrenia, traumatic brain injury, an eating disorder, a disorder of consciousness, and an autistic spectrum disorder;
operating an implantable pulse generator to generate electrical pulses; and
providing the electrical pulses from the implantable pulse generator to tissue of the posterior cingulate to treat the neurological disorder.

13. The method of claim 12 wherein the eating disorder is selected the list consisting of: obesity and anorexia nervosa.
